(19) United States
(12) Patent Application Publication
(10) Pub. No.: US 2011/0245713 A1
(43) Pub. Date: Oct. 6, 2011

(54) MEDICAL DEVICE COMPRISING A PROBE FOR MEASURING TEMPERATURE DATA IN A PATIENT'S TISSUE

(75) Inventors: Judith Margreet Rensen, Eindhoven (NL); Jaap Haartsen, Eindhoven (NL); Mareike Klee, Eindhoven (NL); Drazenko Babic, Eindhoven (NL); Bernardus Hendrikus Wilhelmus Hendriks, Eindhoven (NL); Christianus Martinus Van Heesch, Eindhoven (NL); Bout Marcelis, Eindhoven (NL)

(73) Assignee: KONINKLIJKE PHILIPS ELECTRONICS N.V., EINDHOVEN (NL)

(21) Appl. No.: 13/127,046

(22) PCT Filed: Nov. 9, 2009
(86) PCT No.: PCT/IB2009/054965

§ 371 Date: Apr. 30, 2011

(30) Foreign Application Priority Data
Nov. 11, 2008 (EP) 08168850.9

(51) Int. Cl. A61B 5/01

(52) U.S. Cl. 600/549

(57) ABSTRACT

A medical device comprising a probe for measurement temperature data of tissue within a patient's body is proposed. The probe (2) comprises a flexible substrate (3) attached to a medical device core (5), the flexible substrate (3) comprising one or more thermopiles (7) and may furthermore comprise resistors for measuring an absolute temperature and heat sources for locally applying heat. The thermopiles can be processed directly on a flexible polymer carrier or, alternatively, on a silicon substrate and transferred to a flexible carrier (3) enabling both, a highly flexible substrate (3) and very small structural dimensions for the thermopiles (7) and, possibly, the resistors and heat sources. Accordingly, measurement of temperature gradients of tissue being in contact to the medical device may be performed at high resolution allowing reliable detection of temperature anomalies e.g. due to malign tissue.

### FIELD OF THE INVENTION

The present invention relates to a medical device comprising a probe for measuring temperature data such as temperature gradients, thermal conductivity or thermal capacity in a patient's tissue.

### BACKGROUND OF THE INVENTION

In health care, it may be beneficial to be able to discriminate between different tissues during various procedures. An example of such procedure is a biopsy. Biopsies can fail because samples are not taken at the right location. Means to distinguish between healthy and malignant tissue during biopsies can be helpful to check if samples are taken at the right place. In this way, the number of successful biopsies can be increased. Another example is the treatment of inflamed tissue. In some cases, drugs need to be administered at the location of the inflammation, e.g. during the treatment of low back pain caused by an inflammation. If the drugs are released at the wrong location the treatment is not effective. Techniques to improve the localization of affected tissue therefore will improve the treatment of inflamed tissue. A third example is to distinguish ablated tissue from non-ablated tissue during ablation procedures. Distinction may be useful to monitor the process of ablation and to verify whether the targeted tissue has been completely ablated.

Thermal behavior like temperature gradients, thermal conductivity and thermal capacity of a patient's tissue may be used to distinguish between different tissue types. The temperature of tumor tissue is for example known to be approximately 0.5° C. up to 1.8° C. higher than that of unaffected surrounding tissue. The thermal conductivity of tissue may drop for instance from 0.61 to 0.50 Wm⁻¹K⁻¹ after ablation.

In the prior art, it is known to provide medical instruments with means for measuring temperature data of tissue in a patient's body. However, such prior art approaches may suffer from insufficiencies such as a need for a complex structural arrangement or attachment of temperature measuring means to an actual core of a medical device, insufficient temperature measuring accuracy, a restriction to a limited number of temperature measuring types or high costs for the production of such medical instruments.

### SUMMARY OF THE INVENTION

Accordingly, there may be a need for a medical device comprising a probe for measuring temperature data in a patient's tissue which may allow to overcome at least some of the prior art insufficiencies. Particularly, there may be a need for a medical device comprising a probe for measuring temperature data in a patient's tissue wherein the probe may be easily and cheaply fabricated and may be easily attached to a core of the medical device and which furthermore allows accurate, reliable and/or fast measurement of one or more temperature data types such as temperature gradients, thermal conductivity and thermal capacity.

These needs may be met by the subject-matter according to the independent claim. Advantageous embodiments of the present invention are described in the dependent claims.

According to a first aspect of the present invention, a medical device comprising a probe for measuring temperature data of tissue within a patient's body is proposed. Therein, the probe comprises a flexible substrate attached to a medical device core, the flexible substrate comprising one or more thermopiles.

A gist of the present invention may be seen as being based on the following ideas:

The invention relates to medical devices such as needles, scopes, catheters and any other surgical tools with a new type of temperature sensor or temperature probe to determine tissue differences such as inflamed regions or cancerous tissue. Therein, the sensor or probe includes a flexible substrate comprising an array of thermopiles which, as will be explained in further detail further below, may be fabricated by integration on a silicon substrate and transfer to a flexible polymer carrier using for example circonflex technology as an enabling technology. The sensor or probe may be applied to a core of the medical device which may comprise e.g. the surgical instrument or a needle, scope, catheter etc.

The use of such specific sensor or probe may have a number of advantages over conventional temperature sensing means: (a) A first advantage may be that thermopiles are substantially offset free. This does not hold for thermistors. This may be especially advantageous in case multiple sensors, i.e. thermopiles, will be used on one probe to compare data from different regions; (b) A second advantage may be the accuracy of such type of sensor or probe. Silicon IC processed thermocouples can be connected electrically in series forming a so-called thermopile. The advantage may be that individual signals (approximately 200 μV.K⁻¹) of single thermocouples may add up thereby increasing the absolute signal and the signal-to-noise ratio. As has been experimentally shown, such thermopiles including for example 175 thermocouples with an area of 8 mm² may detect temperature differences in the range of 10 μK. In contrast to this, the typical accuracy of small thermistors is in the order of 10th of Kelvin; (c) A third advantage may be that such temperature sensors or probes are flexible and can therefore easily be adapted to the shape of a medical instrument which simplifies the integration of probe and medical instrument.

In the following, possible features and advantages of embodiments of the proposed medical device will be described.

The medical device proposed herein may be a combination of a medical instrument forming a core of the medical device and a probe for measuring temperature data. In other words, the probe comprising a plurality of thermopiles may be combined with other functionalities on medical instruments. For example, the probe may be applied to a photonic needle which can be used for thermal and optical detection of tissue. In a second example, the probe may be mounted next to an ultrasound transducer array on a medical instrument such as a catheter or a needle or a scope to enable local ultrasound imaging and measurement of temperature data. A third example may be a medical device that comprises an ultrasound transducer for elasticity sensing to detect malign tissue next to a temperature gradient sensing probe with the thermopiles to determine tumor regions.

The temperature data to be measured by the probe can be different types of data which may depend on the local temperature of the tissue to be examined as well as on temperature differences or gradients wherein the temperature data may be influenced by characteristics of the tissue itself such as a blood circulation there through or an inflammation or tumor state. For example, the temperature data may be a temperature gradient, an absolute temperature, a thermal conductivity in a patient's tissue or a thermal or volumetric heat capacity in a patient's tissue. The temperature data may be localized data, i.e. the temperature data may depend on the location where they are measured and may vary from site to site.

The flexible substrate comprising the thermopiles may be any substrate which has a sufficient flexibility in order to be attached to preferably comply with a surface of the medical device core, i.e. a medical instrument, with which the probe shall be combined. For example, the substrate should be flexible enough for bending to a curvature radius of less than 5 cm, preferably less than 1 cm and more preferably less than 2 mm. Furthermore the flexible substrate may have an extremely low thermal conductivity to avoid heat flow, which may reduce the measurement accuracy.

The thermopiles included in the flexible substrate may comprise a plurality of thermocouples. A thermocouple may consist of two metal or semiconductor structures of different composition a and b that are connected at one end. If the temperature at the junction is raised while the remaining opposite ends are kept at a lower temperature, an open circuit voltage will be measured between the remaining ends. In electrical engineering and industry, thermocouples are a widely used type of temperature sensor and can also be used as a means to convert thermal potential difference into electric potential difference. Such thermocouples which operate based on the so-called thermoelectric effect or Seebeck effect may be cheap and simple devices being able to measure the temperature difference between two points, namely a hot end and a cold end. Usually, the open circuit voltage increases with increasing temperature difference and can typically be between 1 and 70 μV per degree Celsius for metals and 1-1000 μV per degree Celsius for semiconducting materials.

Connecting a number of thermocouples in series may form a so-called thermopile of which the open circuit voltages can be added which makes a thermopile a very sensitive offset free temperature difference sensor. Accordingly, a thermopile may be seen as an electronic device that converts thermal energy into electrical energy. The thermopile does not measure an absolute temperature but generates an output voltage depending on a local temperature difference or a temperature gradient. The output voltage of the thermopile may usually be in the range of tenth or hundreds of mV. It is both possible to equip a probe with a single thermopile or with an array of thermopiles. The latter option makes it possible to obtain information on thermal properties along a surface of the medical device or along a path of the medical device without having to displace the medical device.

According to an embodiment of the present invention, a plurality of thermopiles are arranged on the flexible substrate of the probe wherein the thermopiles are spaced from each other at distances of less than 5 millimeters, preferably less than 1 mm and more preferably less than 0.5 mm. In other words, the plurality of thermopiles is arranged as an array in which the respective thermopiles are arranged very close to each other. The array of thermopiles thereby covers a surface in which the temperature data may be measured wherein each thermopile may measure a temperature gradient between a first contact surface of the thermopile being in contact with a low temperature site and a second contact surface of the thermopile being in contact with a high temperature site. Therein, the distance between adjacent thermopiles may be interpreted as the distance between the geometrical centers of the entire contact surface of the respective thermopiles. The distance between the thermopiles can be adapted to the structural sizes of the tissue to be examined. For example, if a tissue structure such as a tumor having a size of a few mm is expected to be measured, the distance between neighboring thermopiles may be in the same order of magnitude, namely a few mm or less. Accordingly, temperature data such as temperature gradients may be measured at high resolution allowing detecting small sized tissue anomalies.

According to a further embodiment of the present invention, at least one of the thermopiles on the substrate has a contact surface of less than 40 mm², preferably less than 4 mm² and more preferably less than 1 mm² and even more preferably less than 0.1 mm². Each thermopile may comprise more than 10, preferably more than 50 and more preferably more than 100 thermocouples connected in series. Therein, the contact surface of the thermopile may be interpreted as the surface to be in contact with the local tissue area to be measured including the high temperature site and the low temperature site. It may be advantageous to prepare the thermopiles with an as small contact surface as technically possible. The smaller the contact surface, the smaller the area in which a temperature gradient can be measured and, accordingly, the smaller the area or size in which a tissue anomaly can be detected.

According to a further embodiment of the present invention, the flexible substrate comprises a flexible polymer substrate. For example, a flexible carrier such as a foil of polyimide, teflon or any other organic material may be used. On top of this carrier the thermocouples may be processed using deposition and patterning of metal layers, semiconducting layers or polymer semiconductor layers. Very small structures having structural dimensions in the range of a few μm or less may be generated using for example photolithography processes. The total thickness of the flexible carrier with the thermopiles may be dependent on the application and may be in the order of 200 μm down to less than 20 μm and more preferably less than 10 μm thereby allowing for the necessary flexibility of the substrate.

According to a further embodiment of the present invention, the flexible substrate comprising the thermopiles is produced by generating conducting structures using silicon technology and then transferring the conducting structure to the flexible substrate. Such processing may also be referred to as so-called circonflex technology. In circonflex technology, circuits comprising conducting structures including metal or semiconducting structures may be fabricated on SOI (Silicon On Insulator) wafers. Alternatively also silicon wafers with a thermal silicon oxide layer may be used. Using SOI wafers the thermopiles may be processed in doped mono-crystalline or polycrystalline silicon. Alternatively for the thermopiles also metals or combinations of silicon with metals such as Al may be used. In case that the device should comprises only the thermopiles and possibly also resistors and/or heating elements but no electronics, also a silicon substrate with a thermal oxide layer can be used. Then also the thermopiles may be processed in doped polycrystalline silicon or from metals or combinations of polysilicon with metals such as Al. To achieve small features typically photolithographic processes may be applied. To realize a flexible device, polymers such as polyimide or any other system such as biocompatible parylene may be applied on top and the Si wafer with the polyimide may be temporarily glued on a carrier. The silicon may be etched away from the back-side using the silicon oxide as an etch stop. To contact the thermopiles the SiO₂ may be opened with lithographic processing and finally the glass is removed.

In other words, the thermopiles and optionally also heat sources and/or resistors and further optionally also electronics for data acquisition, data processing and/or wireless data transfer may be processed on silicon technology and may then be transferred to a flexible carrier in a post-processing step where non-functional silicon is fully removed.

Details of a circonflex technology are described in U.S. Pat. No. 6,762,510 the content of which is incorporated herein by reference.

Highly flexible circuits may remain defect-free even after bending to a radius of curvature of less 1 mm. The circuits fabricated on the SOI wafer may be produced using silicon technologies which allow for both, high reliability and very small structure sizes. The devices achieved in this way show a low thermal conductivity, high accuracy and small feature size, so that a large amount of thermocouples can be designed in parallel on a small area.

The circonflex approach has the further advantage that electronics for data acquisition, data processing and wireless data transfer can be processed in the silicon. The polymer carrier in the circonflex enables an efficient RF performance for wireless data transfer.

According to a further embodiment of the present invention, the flexible substrate is wound around the probe core. Due to its flexibility, the substrate comprising the thermopiles may easily comply with the surface of the probe core, namely the underlying medical instrument. By winding the flexible substrate around a probe core, the thermopiles arranged on the substrate may be easily attached to the surface of the probe core.

According to a further embodiment of the present invention the probe is adapted to measure at least one of an absolute temperature and temperature gradients along tissue region of interest. Accordingly, temperature variation along a tissue region may be measured in order to locate regions of locally strongly varying temperatures which may indicate e.g. malign tissue. Furthermore, these measurements may be calibrated using a measurement of an absolute temperature.

According to a further embodiment of the present invention probe further comprises at least one resistor adapted for absolute temperature measurement. For example, the substrate comprising the thermopiles may be combined with thin film resistors. With the thin film resistors which can be for example a thin metal layers with defined length, width and height along the temperature dependence of the resistor the absolute temperature of the tissue where the probe is positioned may be determined. This absolute temperature determined with at least a minimum of one resistor combined with the temperature gradient measurements of several thermopiles enables the determination of the absolute temperature along the tissue region of interest.

According to a further embodiment of the present invention, the probe furthermore comprises one or more heat sources. For example, a heat source may be provided adjacent to a thermopile. Each thermopile may have its corresponding own heat source. For example, a resistive wire element may serve as a heat source upon application of an electrical voltage. The heat source/sources may enable the measuring of a thermal conductivity or a thermal capacity of the underlying tissue. Therein, the heat source may serve for locally heating the underlying tissue and the thermopile may be used to detect a temperature gradient in the tissue resulting from such local heating. The heat source may be combined with a single thermopile on the probe or on every thermopile on the probe. In this latter case, information on the thermal conductivity and the thermal capacity can be obtained along a respective surface or a respective path of the medical device without having to displace the medical device. It may be advantageous if the thermopiles are distributed at such distances from each other that the heat sources of the different thermopiles do not cause interference of measurement signals.

According to a further embodiment of the present invention, the heat source/sources is/are integrated in the flexible substrate. Particularly in case that the flexible substrate is a silicon substrate both, the thermopile(s) and the heat source(s) may be produced using the same technologies, i.e. silicon technologies and, preferably, may be produced in same processing steps. Furthermore, the heat source(s) may be formed with a dimensional size corresponding to the dimensional size of the thermopile(s).

According to a further embodiment of the present invention, the probe is adapted to measure a thermal conductivity in a patient's tissue. For this purpose, a heat source and a thermopile may be provided. The heat source temporarily heats the patient's tissue locally and the thermopile may be used to determine how the heat spreads over the patient's tissue by monitoring a temperature gradient in the neighborhood of the heat source. From the variation of the temperature gradient, being influenced both by the position and the time of the measurement, conclusions can be drawn with respect to the local thermal conductivity of the patient's tissue. There from, further conclusions can be drawn with respect to other characteristics of the patient's tissue such as for example its density or water-content which then may provide information for example on its malignity.

According to a further embodiment of the present invention, the probe is adapted to measure the volumetric heat capacity in a patient's tissue. Again, a heat source and a thermopile may be provided. After temporarily and locally heating the patient's tissue, the thermopile may be used to determine the reaction of the tissue, namely the spreading of the provided heat over the time. There from, information on the thermal capacity of the underlying tissue may be derived which then again may provide information on further characteristics on the patient's tissue.

According to a further embodiment of the present invention, the probe is thermally isolated against the medical device core, namely the underlying medical instrument being mechanically coupled with the temperature probe. Such thermal isolation may prevent an undesired heat transport from the medical device core to the temperature probe. The thermal isolation may be provided by a separate isolating layer interposed between the medical device core and the substrate carrying the thermopiles. Alternatively, the probe itself may be made with a heat isolating synthetic material. The thermal isolation may reduce the thermal influence of the medical core device to the probe which otherwise might disturb the probes temperature measurements.

According to a further embodiment of the present invention, the probe is adapted for wireless transmission of data on temperature data measured in a thermopile. In other words, the probe may include means for wirelessly transmitting information about the temperature data measured by one of the piles or each of the plurality of piles of the probe. Therein, the wireless data transmission may occur between a transmitter in the probe and a receiver included within the medical device core which then may transmit the temperature data to an analyzing device connected thereto and being positioned outside the patient. Alternatively, the wireless data transmission may occur directly between the probe and the outside analyzing device. Using wireless data transmission between the probe and a spaced apart receiver may omit the necessity of direct wiring to the probe wherein otherwise such direct wiring may act as a thermal bridge for example between the medical device core and the temperature probe.

It has to be noted that features and advantages of the present invention have been described with reference to different embodiments of the invention and, partly, also with respect to manufacturing process for the inventive device. However, a person skilled in the art will gather from the above and the following description that, unless other notified, in addition to any combination of features belonging to one embodiment also any combinations between features relating to different embodiments or to a manufacturing method is considered to be disclosed with this application.

### BRIEF DESCRIPTION OF THE DRAWINGS

Features and advantages of the present invention will be further described with respect to specific embodiments as shown in the accompanying figures but to which the invention shall not be limited.

FIG. 1 shows a thermocouple.

FIG. 2 shows details of a medical device comprising a temperature measurement probe according to an embodiment of the present invention.

FIG. 3 shows an arrangement of a biopsy needle comprising several temperature probes according to an embodiment of the present invention upon arrangement on a hot spot within a patient's tissue.

FIG. 4 shows a specific embodiment of a temperature measuring probe including a heat source for a medical device according to an embodiment of the present invention.

FIG. 5 shows the distribution of a temperature and a temperature gradient depending on a position on a line through a tumor.

The drawings in the figures are only schematically and not to scale. Similar elements in the figures are referred to with similar reference signs.

### DETAILED DESCRIPTION OF PREFERRED EMBODIMENTS

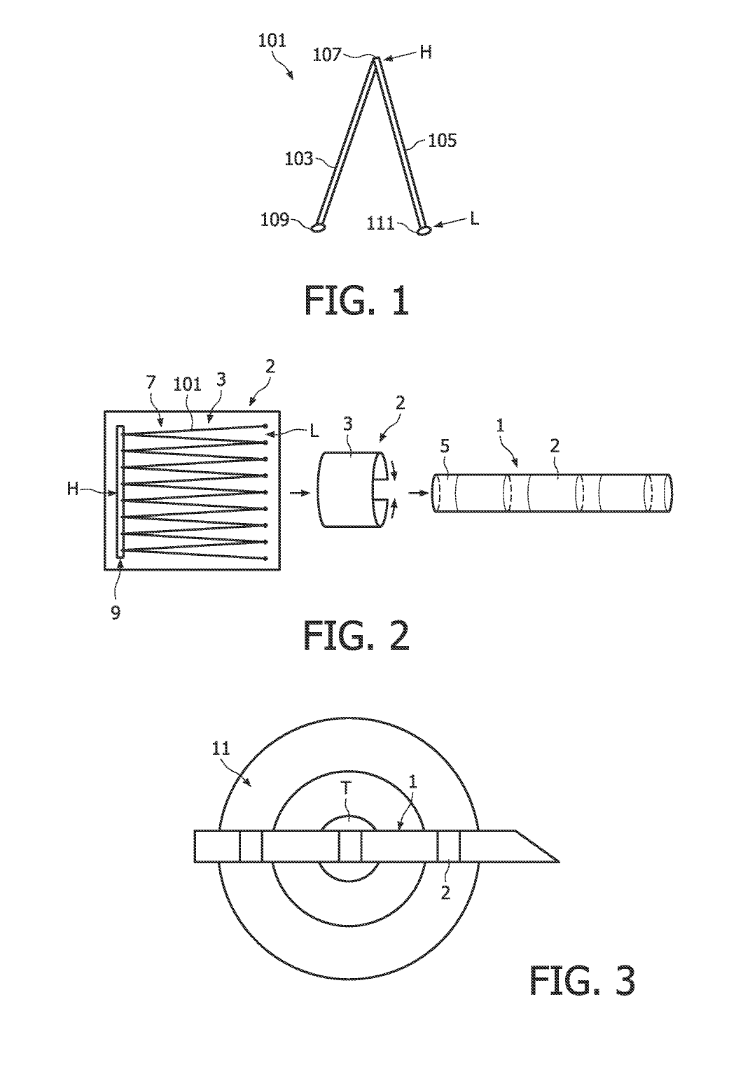

FIG. 1 shows a thermocouple 101 in principle. Two electrically conductive structures in the form for example of wires 103, 105 are provided. The material of the wires 103, 105 can be metals or semiconductors. According to the thermoelectric effect, also known as Seebeck effect, when a conductor is subjected to a thermal gradient, it will generate a voltage between its ends. Any attempt to measure this voltage necessarily involves connecting another conductor to the “hot” end. This additional conductor will then also experience the temperature gradient and develop a voltage of its own which will oppose the original voltage. The magnitude of the effect depends on the material in use. Using different materials for the respective wires 103, 105 to complete the circuit creates a circuit in which the two legs generate different voltages, leaving a small difference in voltage available for measurement. While a first end 107 at which the two wires 103, 105 are connected to each other is placed at a site with a first temperature, for example at a high temperature site H, the other ends of the wires 103, 105 are positioned at a site being on a different temperature, for example a low temperature site L. Due to the thermoelectric effect, an open circuit voltage V can be measured between terminals 109, 111 at the respective ends of the wires 103, 105. The open circuit voltage increases with the temperature and may therefore give an indication on a temperature difference between the high temperature site and the low temperature site.

In FIG. 2, the arrangement of a medical device 1 such as for example a biopsy needle is schematically shown. A thermopile 7 comprising a plurality of thermocouples 101 similar to the thermocouple 101 shown in FIG. 1 and being connected in series is implemented in a flexible substrate 3 in order to form a temperature probe 2. The flexible substrate comprises a thin polymer carrier or the circonflex carrier (where the stack has been described above) having a thickness of for example 10 μm. On the carrier conducting lines corresponding to the wires 103, 105 have been prepared using conventional photolithographic technologies such as photolithography thereby allowing very small structural dimensions for the thermopile 7. For example, the entire thermopile 7 can be prepared on a surface of the substrate 3 within an area of less than 1 mm².

Additionally, a heater serving as a heat source 9 is arranged on the substrate 3 at a position in the neighborhood to one end of the thermopile. Using the heat source 9, tissue adjacent to the substrate 3 may be locally heated.

Due to its high flexibility, the substrate 3 may be wound around the tip of the medical device 1, i.e. the biopsy needle. Therein, several substrates 3 comprising a plurality of thermopiles 7 may be attached to the core of the biopsy needle serving as a medical device core 5. Using all these thermopiles as temperature sensors, local temperature gradients along the tip of the biopsy needle may be measured.

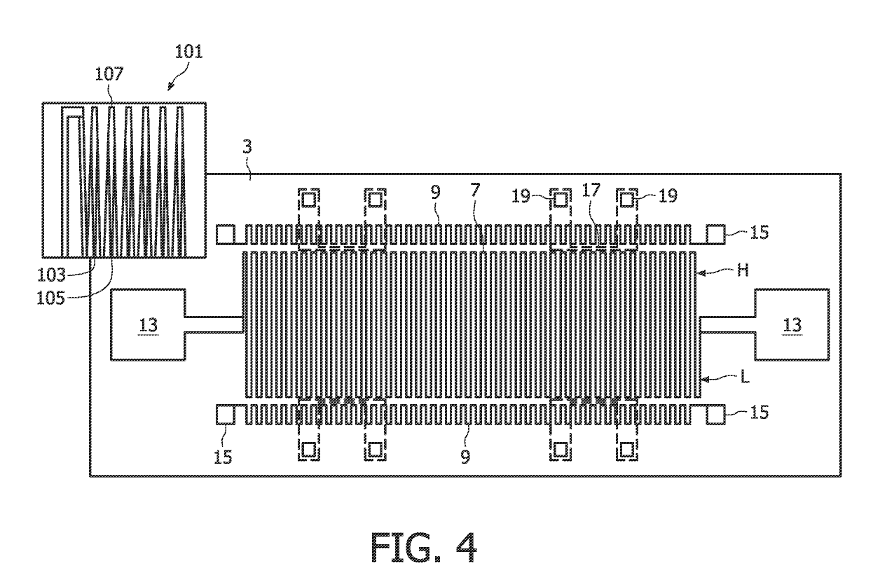

FIG. 3 schematically shows the arrangement of a biopsy needle 1 comprising three temperature measuring probes 2 positioned at different locations along a longitudinal extension of the needle. The surface of the needle 1 is in mechanical contact with the surrounding patient's tissue 11. Within this surrounding tissue 11, a tumor T might be present. Accordingly, the needle 1 may be positioned such that it traverses the tumor tissue T. The temperature distribution in the normal tissue 11 differs from the temperature within the tumor tissue T. Furthermore, other characteristics such as the thermal capacity and the thermal conductivity may depend on the type of the tissue, malign or not. Accordingly, using the probes 2 attached to the surface of the needle 1, a distribution of temperature gradients as well as of the local thermal capacity or thermal conductivity may be measured.

It is to be noted that in FIG. 3 the arrangement of the temperature measuring probes 2 as well as their size is only represented schematically. Of course, possible substrates 3 comprising thermopiles 7 and acting as probes 2 may be realized much smaller than represented in the drawings and may be arranged on the surface of the medical device 1 much more closely to each other than represented. Accordingly, a distribution of the measured temperature data may be acquired with high resolution.

FIG. 4 shows an example of a probe 2 prepared using circonflex technology. On a flexible thin film substrate 3, a thermopile arrangement 7 comprising a multiplicity of for example one hundred thermocouples 101 is arranged. As can be seen in the magnified portion of FIG. 4, conducting wires 103, 105 of different materials with typical dimensions of a length of a few mm down to several hundred micrometer and width of several micrometer up to several tens of micrometer are prepared such as to overlap in regions 107 thereby forming a single thermocouple 101. While first lapping regions 107 are arranged on a high temperature site H of the probe 2, opposing ends of the wires 103, 105 are arranged on a low temperature site L. Respective ends of the series connection of thermocouples 101 forming the thermopile 7 are connected to terminals 13.

The probe 2 comprises on the surface of the substrate 3 two additional heaters as optional heat sources 9. The heaters are provided close to the respective sides of the thermopile 7 at the high temperature site H and the low temperature site L and may be used to heat a respective local region. Accordingly, the high temperature site H may be heated using the heat source 9 adjacent thereto while the low temperature site L may remain at the original temperature as long as the heat source 9 adjacent thereto is switched off. Of course, this temperature arrangement can also be inversed by changing the switching states of the respective heat sources 9. Each of the heat sources 9 can be provided by a resistive wire pattern which, at its respective ends, is connected to terminals 15.

Furthermore, resistors 17 are provided. These resistors 17 are adapted to measure absolute temperature at locations adjacent to the thermopile 7. The resistors 17 are connected to bond pads 19 at which an electrical signal of the resistors can be gripped.

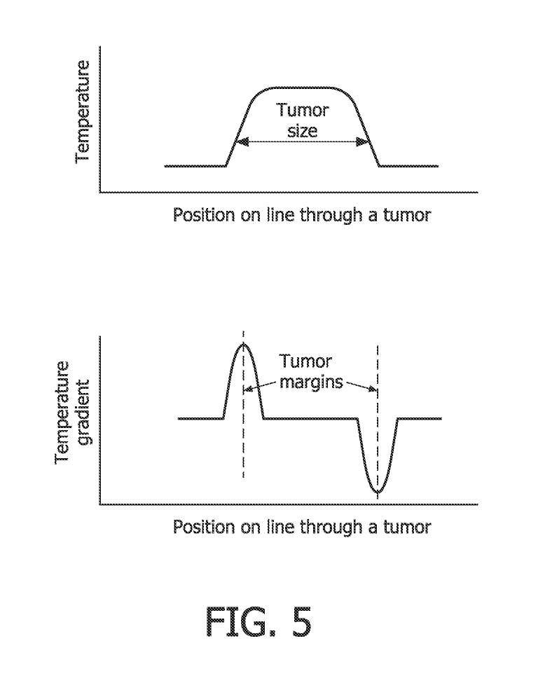

Referring to FIG. 5, a measuring procedure using the medical device according to the present invention will be explained. On the upper graph in FIG. 5, the local temperature dependent on the position within a malign tissue is shown. While at normal tissue the temperature is constantly at a first, lower level, in a region adjacent to a tumor the temperature is locally increased. As can be seen in the lower graph of FIG. 5 representing the dependence of the temperature gradient on the proposition on a line through the tumor, the temperature gradient is particularly emphasized on the margins of the tumor.

Using the medical device according to the present invention having a temperature measuring probe at its surface, such increased temperature gradients can be measured locally at a high resolution thereby giving precise information about the local margins of malign tissue.

Finally, different measurement methods which may be applied using the medical device according to embodiments of the present application are briefly described.

A temperature gradient may be determined by applying passive measurements, while a thermal conductivity or a thermal capacity can only be determined by active measurements.

First, the passive measurements shall be exemplary described. A single thermopile may give information on the local temperature gradient in tissue being in contact to the thermopile. This type of measurement may be used to determine temperature fluctuations but also to determine the margins of lesions.

For example, such passive temperature gradient measurement may be used to determine precise locations and margins of tumors as described further above with respect to FIG. 5.

Active measurements comprise the measurement of thermal conductivity or of thermal capacity. The thermal conductivity can be determined from the temperature gradient which the thermopile is measuring along its dimensions when the heater 9 is set to a constant slightly higher temperature than the surrounding tissue. The relation between the temperature gradient, the thermal conductivity and the applied heat is given by a heat equation that in the steady case is given by

∇•(κ∇T)=−q

wherein T is the temperature, q is the heat flux and κ is the thermal conductivity.

The thermal conductivity and the thermal capacity can be determined by performing measurements on the dynamic behavior of the tissue. The dynamic behavior can be described by a heat equation that in the transient case reads as

$$ρ {c}_{p} \frac{d T}{d t} - Δ \cdot ( κ Δ T ) = q$$

where ρ is the density and cₚ is the specific heat.

Finally, some approaches to measure the dynamic behavior are proposed: (a) A time varying signal such as a sine, block function, may be applied to the heater 9 and the phase shift which depends on ρ, cₚ and κ is measured; (b) A heat pulse is applied and the time it takes the heat pulse to travel a distance along the probe (time of flight) is measured which can be measured with the thermopiles.

The medical device according to embodiments of the present invention may be used in biopsy procedures, during treatment of inflammation or for monitoring the effect of ablation during an ablation procedure. In an extension of the present invention, the thermopiles may be applied to a photonic needle which can be used for thermal and optical detection. In a further extension of the present invention, the thermopiles can be combined with ultrasound transducers on a probe. Both extensions may enable to perform multi-parameter measurements which may increase the reliability of such measurements.

Summarizing, a medical device comprising a probe for measurement temperature data of tissue within a patient's body has been presented. The probe 2 comprises a flexible substrate 3 attached to a medical device core 5, the flexible substrate 3 comprising one or more thermopiles 7 and may furthermore comprise resistors 17 for measuring an absolute temperature and heat sources 9 for locally applying heat. The thermopiles can be processed directly on a flexible polymer carrier or, alternatively, on a silicon substrate and transferred to a flexible carrier 3 enabling both, a highly flexible substrate 3 and very small structural dimensions for the thermopiles 7 and, possibly, the resistors 17 and heat sources 9. Accordingly, measurement of temperature gradients of tissue being in contact to the medical device may be performed at high resolution allowing reliable detection of temperature anomalies e.g. due to malign tissue.

Finally, it should be noted that the terms “comprising”, “including”, etc. do not exclude other elements or steps and the terms “a” or “an” do not exclude a plurality of elements. Also, elements described in association with different embodiments may be combined. It should also be noted that reference signs in the claims should not be construed as limiting the scope of the claims.

1. A medical device (1) comprising a probe (2) for measuring temperature data of tissue (11) within a patient's body, the probe (2) comprising:
a flexible substrate (3) attached to a medical device core (5), the flexible substrate (3) comprising one or more thermopiles (7).

2. The medical device of claim 1,
wherein a plurality of thermopiles (7) are arranged on the flexible substrate (3) wherein the thermopiles are spaced from each other at distances of less than 5 millimeters.

3. The medical device of claim 1,
wherein at least one of the thermopiles (7) on the substrate (3) has a contact surface of less than 40 mm².

4. The medical device of claim 1,
wherein the flexible substrate (3) comprises a polymer substrate.

5. The medical device of claim 1,
wherein the flexible substrate comprising the thermopiles is produced by generating conducting structures using silicon technology and then transferring the conducting structure to the flexible substrate.

6. The medical device of claim 1,
the flexible substrate (3) is wound around the medical device core (5).

7. The medical device of claim 1,
wherein the probe (2) is adapted to measure at least one of an absolute temperature and temperature gradients along tissue region of interest.

8. The medical device of claim 1,
wherein the probe further comprises at least one resistor (17) adapted for absolute temperature measurement.

9. The medical device of claim 1,
wherein the probe (2) furthermore comprises a heat source (9).

10. The medical device of claim 9,
wherein the heat source (9) is integrated in the flexible substrate (3).

11. The medical device of claim 1,
wherein the probe (2) is adapted to measure a thermal conductivity in a patient's tissue.

12. The medical device of claim 1,
wherein the probe (2) is adapted to measure volumetric heat capacity in a patient's tissue.

13. The medical device of claim 1,
wherein the probe (2) is thermally isolated against the medical device core (5).

14. The medical device of claim 1,
wherein the probe (2) is adapted for wireless transmission of data on temperature measured in a thermopile (7).
